(19) United States
(12) Patent Application Publication
(10) Pub. No.: US 2014/0081208 A1
(43) Pub. Date: Mar. 20, 2014

(54) INJECTION PRESSURE MONITORING DEVICE AND SYSTEM

(71) Applicant: ISTO Technologies, Inc., St. Louis, MO (US)

(72) Inventor: Michael Maloney, St. Louis, MO (US)

(21) Appl. No.: 14/116,951

(22) PCT Filed: May 11, 2012
(86) PCT No.: PCT/US2012/037548

§ 371 Date: Nov. 11, 2013

Related U.S. Application Data
(60) Provisional application No. 61/484,848, filed on May 11, 2011.

(51) Int. Cl. A61M 5/48

(52) U.S. Cl. CPC A61M 5/486
USPC 604/121

(57) ABSTRACT

The present disclosure describes an injection pressure monitoring device including a pressure sensor, an injection pressure monitoring system and kits for monitoring injection pressure generated at a needle tip when injecting a therapeutic fluid composition into tissue, to avoid generating potentially damaging pressure in the tissue being treated.

### CROSS REFERENCE TO RELATED APPLICATIONS

This application claims priority to U.S. Provisional Application No. 61/484,848, filed on May 11, 2011, and entitled “INJECTION PRESSURE MONITORING DEVICE AND SYSTEM,” the disclosure of which is incorporated herein by reference in its entirety.

### TECHNICAL FIELD

The present disclosure relates generally to medical injection devices and methods, and in particular to medical injection devices and methods for monitoring and controlling injection pressures during injection of a fluid therapeutic composition into tissue of a subject being treated.

### BACKGROUND OF THE INVENTION

Recent advances in cell-based medical therapies include the development of an injectable, cell-based fluid composition that can be injected into damaged or diseased tissue to repair the tissue. For example, an injectable cell-based fluid composition can be used for resolving back pain associated with degenerative disease of the intervertebral disc. The treatment involves the combination of two components in a single fluid stream for injection directly into the disc, via a spinal injection needle placed in the target intervertebral disc. However, the injection pressure at the needle-tip can, if excessive, cause further damage to already compromised tissue. To avoid further damage to already compromised tissue, injection pressure must somehow be monitored and controlled. Various approaches to doing so are theoretically possible, but pressure cut-offs are not clear, and certain monitoring/control approaches can further complicate treatment by reducing the accuracy of fluid delivery to the tissue. Devices and methods for monitoring and controlling injection pressure for injection of a therapeutic fluid composition are therefore needed.

### SUMMARY OF THE INVENTION

In one aspect, the present disclosure provides an injection pressure monitoring device for coupling to a dispensing syringe, which comprises a pressure sensor comprising a miniature or subminiature load cell operable for measuring a compression load of up to about 100 lb, having an amplifier and an electrical coupling coupled thereto for coupling to a pressure display unit; a syringe adapter coupled to the load cell, the syringe adapter having a first surface configured to engage an outward end portion of a plunger rod of a plunger rod assembly of the dispensing syringe; and a finger plate coupled to the load cell. The injection pressure monitoring device can be configured for coupling to a dispensing syringe that is for example a double-barreled syringe having a plunger rod assembly comprising two plunger rods each having an outward end portion, and an end plate coupling the outward end portions of the two plunger rods, wherein the syringe adapter has a surface configured to engage the end plate of the plunger rod assembly. In the injection pressure monitoring device, the load cell is for example a subminiature load cell operable for measuring a compression load of up to about 50 lb. The load cell can be, for example, a universal (tension/compression) or a compression only load cell. The finger plate can be for example an injection molded or machined finger plate. The finger plate can be made of a material such as polytetrafluoroethylene, and the finger plate has a contact surface that may have for example a central depression or finger groove for comfortably positioning a finger when the sensor is in operation with a dispensing syringe.

In another aspect, the present disclosure provides an injection pressure monitoring system for maintaining injection pressure at a predetermined level in a tissue being injected with a fluid therapeutic composition from a dispensing syringe, the system comprising a pressure sensor as described above, a pressure display unit coupled to the pressure sensor electrical coupling and comprising a plurality of alarm elements; and the dispensing syringe, which comprises a syringe body having at least one elongated barrel and a plunger rod assembly comprising a plunger rod having an outward end portion and operable for slidably engaging the inside of the elongated barrel, wherein the pressure sensor syringe adapter is coupled to the outward end portion of the plunger rod. In the system, the dispensing syringe can be a double-barreled syringe having a plunger rod assembly comprising two plunger rods each having an outward end portion, and an end plate coupling the outward end portions of the two plunger rods, wherein the syringe adapter has a surface configured to engage the end plate of the plunger rod assembly. The pressure display unit may comprise a first audible tone alarm and a second audible tone alarm. The pressure display unit may be configured to trigger the first audible tone alarm when the pressure sensor signals a pressure reading of at least 50 psi and to trigger the second audible tone alarm when the pressure sensor signals a pressure reading of at least 60 psi. For example, the pressure display unit may be configured to trigger the first audible tone alarm when the pressure sensor signals a pressure reading of at least 75 psi and to trigger the second audible tone alarm when the pressure sensor signals a pressure reading of at least 95 psi. In the system, the first audible tone alarm maybe an intermittent tone and the second audible tone may be a continuous tone. The system may further comprise a portable DC power source.

In another aspect, the present disclosure provides a kit for monitoring injection pressure in a dispensing syringe during injection of a fluid composition into tissue of a subject, the kit comprising a pressure sensor as described above, and a dispensing syringe having a plunger rod assembly including a plunger rod having an outward end portion. In the kit, the dispensing syringe can be a double-barreled syringe having a plunger rod assembly comprising two plunger rods each having an outward end portion, and an end plate coupling the outward end portions of the two plunger rods, wherein the syringe adapter has a surface configured to engage the end plate of the plunger rod assembly. The load cell can be a subminiature load cell operable for measuring a compression load of up to about 50 lb, and may be for example a compression load cell. The finger plate can be manufactured by injection molding or machining of a polymer such as, for example, polytetrafluoroethylene. The finger plate can be manufactured with a contact surface having a central depression or a finger groove.

### BRIEF DESCRIPTION OF THE DRAWINGS

FIG. 1 is a schematic diagram of a pressure sensor and display unit in an injection pressure monitoring system.

FIG. 2 is a drawing of an injection pressure monitoring device coupled to a double-barreled syringe having a blending connector and spinal needle as configured in an injection pressure monitoring system.

FIG. 3 is a drawing of an overhead side view a syringe adapter in an injection pressure monitoring device.

FIG. 3A is a drawing of a top plan view of the syringe adapter.

FIG. 3B is a drawing of a side plan view of the syringe adapter.

FIG. 4A is a drawing of an overhead side view of the injection pressure monitoring device including a pressure sensor, finger plate, and syringe adapter.

FIG. 4B is a drawing of a side end view of an electrical cable lead on an electrical connector for coupling the injection pressure monitoring device to the display unit.

FIG. 5A is a drawing of a front view of the display unit shown schematically in FIG. 1.

FIG. 5B is a drawing of a back view of the display unit shown schematically in FIG. 1.

FIG. 6 is a schematic diagram of an exemplary circuit for the injection pressure monitoring system showing the pressure sensor (load cell) coupled to a display unit circuit.

FIG. 7 is a drawing of an overhead side view of a calibration clamp used to calibrate the injection pressure monitoring system.

FIG. 7A is a drawing of an overhead view of a horizontal portion of the calibration clamp.

FIG. 7B is a drawing of a side view of the calibration clamp.

FIG. 7C is a drawing of a side view of a vertical portion of the calibration clamp.

FIG. 7D is a drawing overhead side view of a collar used together with the calibration clamp for calibrating the injection pressure monitoring system.

FIG. 8 is a drawing of a complete calibration set-up for calibrating the injection pressure monitoring system, including an injection pressure monitoring device coupled to a double-barreled syringe assembly positioned in the calibration clamp, and a manometer coupled to the syringe assembly via a static fluid line.

FIG. 9 is a drawing of a close-up view of an injection pressure monitoring device including pressure sensor, adapter and finger plate, coupled to double-barreled syringe and positioned in the calibration clamp in preparation for calibration.

### DETAILED DESCRIPTION

An injection pressure measurement device and technique as described herein solves the problem of preventing excessive pressure build-up in a tissue, such as spinal disc tissue, which is being treated by pressure-driven application of a fluid to or into the tissue. Excessive pressure in the tissue may further damage already compromised tissue. The device and technique is useful for example during injection of a fluid therapeutic composition into an intervertebral disc. While a number of approaches to measuring disc pressure are possible and were considered, only the approach as described herein provided suitable pressure monitoring capability while maintaining accuracy of delivery. Several direct measurement techniques were considered and evaluated in cadaver studies. These included use of independent intradiscal pressure measurement devices such as a pressure sensor-tipped catheter and a static fluid line from the disc to a manometer. However, due to the inherent poro-elastic properties of degenerated nucleus pulposus, such measurement techniques did not accurately capture the dynamic injection pressures generated at the injection needle tip. Alternatively, injection line pressures were monitored to capture the highest possible disc pressure during injection. Direct measurement of the injection line was first attempted by means of a static fluid line from the injection line to a manometer through a “T” connection. It was determined however that this set-up would very likely adversely affect accurate delivery of the treatment and it was therefore discarded as an option. Instead, the inventors have succeeded in demonstrating that direct measurement of the force applied to the dual-syringe injection assembly can be suitably calibrated to determine the needle-tip pressure generated by that force. In short, suitable disc pressure can be maintained by monitoring the force applied to the syringe to keep injection line pressures below a predetermined level deemed to be a suitable threshold, above which the risk of further damaging tissue increases significantly. That predetermined level was determined to be about 100 psi.

The present disclosure therefore provides an injection pressure monitoring device, system and related methods for monitoring injection pressure and preventing disc pressure from exceeding the predetermined level.

### A. Definitions

Section headings as used in this section and the entire disclosure herein are not intended to be limiting.

As used herein, the singular forms “a,” “an” and “the” include plural referents unless the context clearly dictates otherwise. For the recitation of numeric ranges herein, each intervening number there between with the same degree of precision is explicitly contemplated. For example, for the range 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9 and 7.0 are explicitly contemplated.

As used herein, the term “monitor” encompasses the acts of measuring and controlling, in this case injection pressure.

### B. Device for Monitoring Injection Pressure

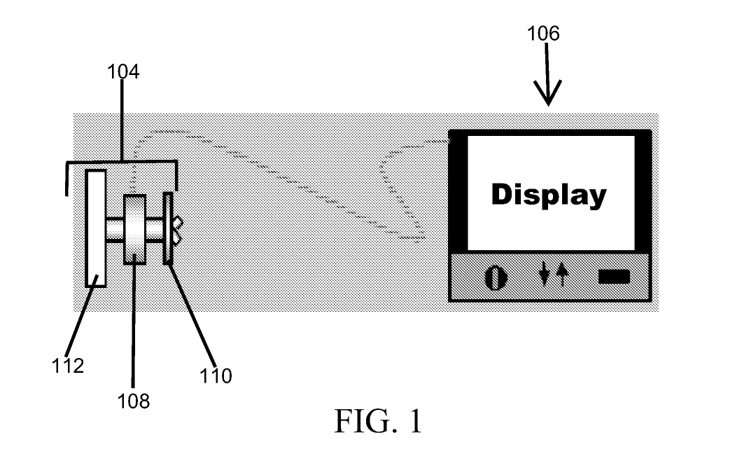

FIG. 1 is a schematic diagram of elements of an injection pressure monitoring system (system) 102. The system 102 includes a pressure sensor 104 coupled to a calibrated display unit 106 having two relay-activated alarms (not shown in FIG. 1). The pressure sensor 104 includes a force transducer 108 positioned between and coupled to, respectively, a syringe adaptor 110 and a finger plate 112 to which pressure is applied digitally, e.g. with a thumb, by the operator.

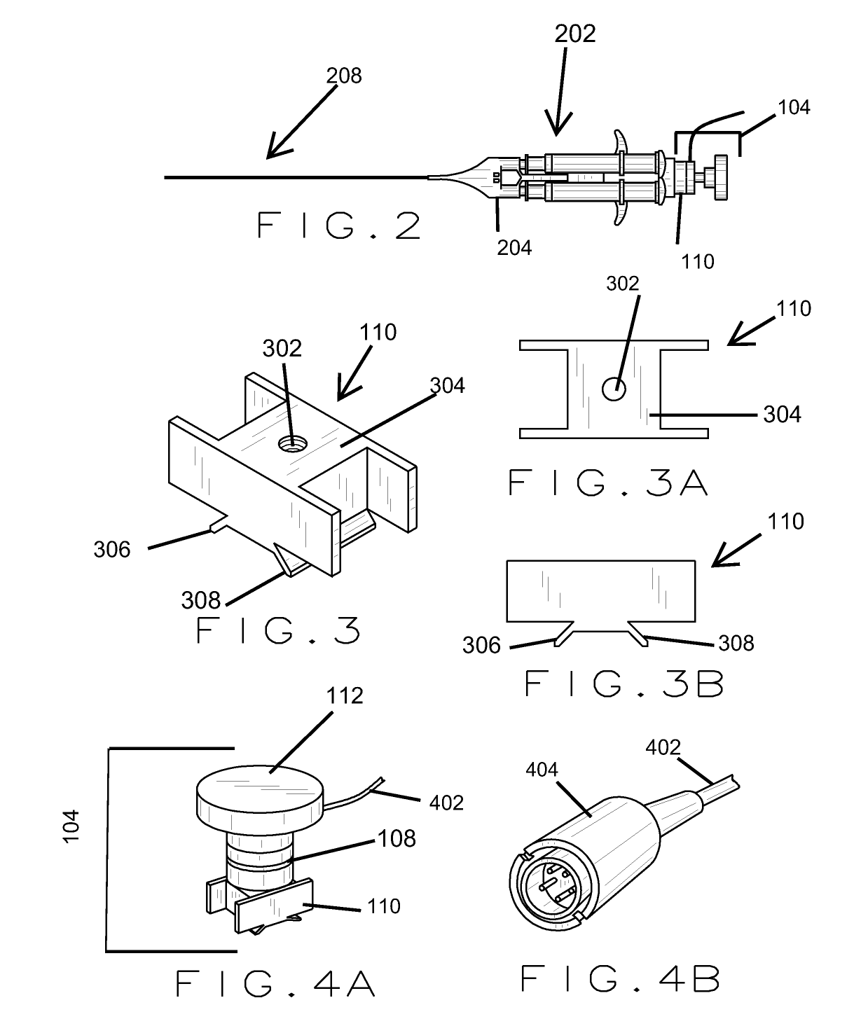

FIG. 2 is a drawing of pressure sensor 104 coupled to a double-barreled syringe (syringe) 202 having a blending connector 204 and spinal needle 208 as configured for use with injection pressure monitoring system 102. As explained in further detail herein below and in the Figures, syringe adaptor 110 is suitably configured for coupling to a pressure-actuated fluid applicator or delivery device such as syringe 202 as shown in FIG. 2. The fluid applicator is used for delivering a fluid therapeutic composition to a damaged intervertebral disc. It will be appreciated that the force transducer 108 is suitably positioned between finger plate 112 and syringe adaptor 110, all of which are positioned along a long axis defined by the finger plate, force transducer and applicator adaptor coupled together in series as shown in the Figures, and further by the length of the applicator, e.g. syringe, to which the pressure sensor is attached, e.g. the long axis of a syringe body.

The applicator is preferably a dispensing or filling syringe as shown in FIG. 2, which includes a syringe body having one or more elongated barrels in each of which a component of the fluid therapeutic composition are placed; and a plunger rod assembly (not shown in FIG. 2) including at least one plunger rod for sliding within the syringe barrel(s). Preferably the dispensing syringe is a double-barreled syringe as known in the art and configured for dispensing a two-component fluid composition, wherein each component is contained separately within one of the two barrels prior to injection, and then the two components combined when simultaneously forced through blending connector 204. An exemplary syringe body is a double-barreled syringe with a plunger rod assembly having two plunger rods, each of which slidably engages within one of the two barrels. Blending connector 204 mixes the two components contained in each barrel, and then directs the mixture through a single syringe needle such as a spinal needle 208 coupled to the syringe body via blending connector 204.

FIGS. 3-3B show different views of an exemplary syringe adapter 110 according to the present disclosure. FIG. 3 is a drawing of an overhead side view of syringe adapter 110, which includes a substantially rectangular body, having a central threaded opening 302 extending along a central axis from a top surface 304 to a bottom surface, syringe-facing surface (not shown) of the body, and two projections 306 and 308, projecting from the bottom surface of syringe adaptor 110. FIG. 3A is a drawing of a top plan view of the syringe adapter showing opening 302 extending through the body of syringe adaptor 110. The dimensions of opening 302 may vary to receive any one of a number of possible threaded coupling elements such as a threaded rod. An exemplary opening 302 is suitably dimensioned and threaded for receiving a #4-40 UNC threaded rod on a load cell, which is described in further detail below. Syringe adaptor 110 may be made using standard engineering design and machining techniques, from any suitably rigid and sterilizable material that can be suitably formed into the adapter shape, e.g., injection molded from plastic or formed from metal sheet or block. An exemplary such material is stainless steel. The materials used for manufacturing all elements of the injection pressure monitoring device are preferably sterilizable by commonly used low-temperature sterilization techniques, such as gas-phase sterilization, including but not limited to low-temperature, hydrogen peroxide gas plasma technology (e.g., STERRAD® Systems), and ethylene oxide sterilization. The adapter is made for example by machining or injection molding, and generally has an oblong shape of a suitable size for maintaining stable contact with the plunger mechanism of the syringe, as shown in FIG. 2.

As shown in FIGS. 3 and 3B, projections 306 and 308 are ridges which project from the bottom surface of syringe adaptor 110 at an approximate 45° angle. While it will be understood that the precise angle of surface projection of the ridges may vary, together they form a dovetail-shaped projection configured to slidably engage with grooves in the cap of a plunger mechanism of a preferred double-barreled syringe, the DuploJect syringe available from Baxter, as described in further detail below.

FIG. 4A is a drawing of an overhead side view of pressure sensor 104, wherein force transducer 108 is a load cell, also including a syringe adaptor 110 and finger plate 112, with electrical cable 402 coupled to force transducer 108. FIG. 4B is a drawing of a side end view of an electrical cable lead 404 on an electrical cable 402 for coupling the injection pressure monitoring device to display unit 106. Finger plate 112 can be molded from any suitably durable and sterilizable material that can be formed into a shape adapted for comfortably receiving and stabilizing a fingertip. Finger plate 112 can be for example machined or injection molded from a polymer such as, but not limited to nylon, polytetrafluoroethylene (PTFE, such as DuPont™ Teflon® brand), acetal and high-performance polyethylene (HPPE). Finger plate 112 may be a substantially planar disc, as shown in FIG. 4A, or the outer surface of finger plate 112 may be adapted, for example manufactured with a depression or groove, for comfortably positioning and stabilizing the finger, e.g. thumb, used for applying pressure to the dispensing syringe when in use for treatment. Finger plate 112 may be coupled to force transducer 108 using any known mechanical or chemical coupling method including but not limited to use of a polymer adhesive, again taking into account the objective of maintaining a mechanically stable connection between the syringe adapter and fingerplate, so that when the device is in use, the coupling is sufficiently robust to prevent force manually applied to the fingerplate from moving off the long axis of the syringe.

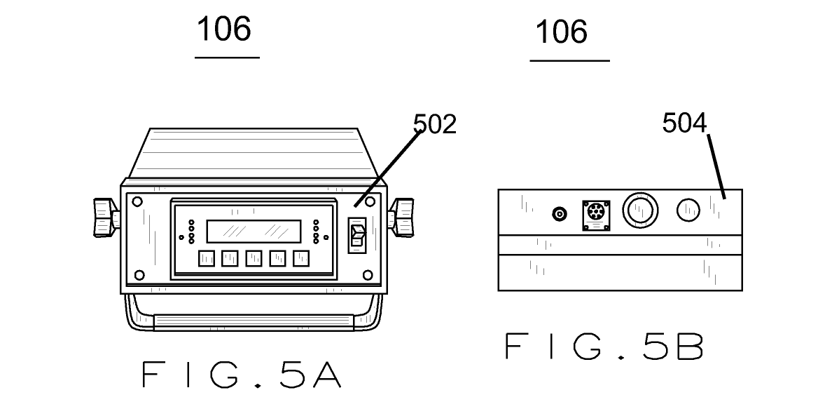

FIG. 5A is a more detailed drawing of a front view of display unit 106 shown schematically in FIG. 1, showing a front surface 502 with control elements including a visual display, LED indicators, function switches and an On/Off switch. FIG. 5B is a drawing of a back view of display unit 106, showing a back surface 504 with electrical coupling elements, including at least one transducer input connector for receiving lead 404. In display unit 106, an internal battery is charged via a power supply input. In use, pressure sensor 104 is further connected via the electrical lead 404 and cable 402 to display unit 106, as shown in FIG. 5B. The display unit is for example a strain gauge panel meter as shown in FIG. 5A, having two relay switches (a DCI 7600A Load Cell Display, or equivalent).

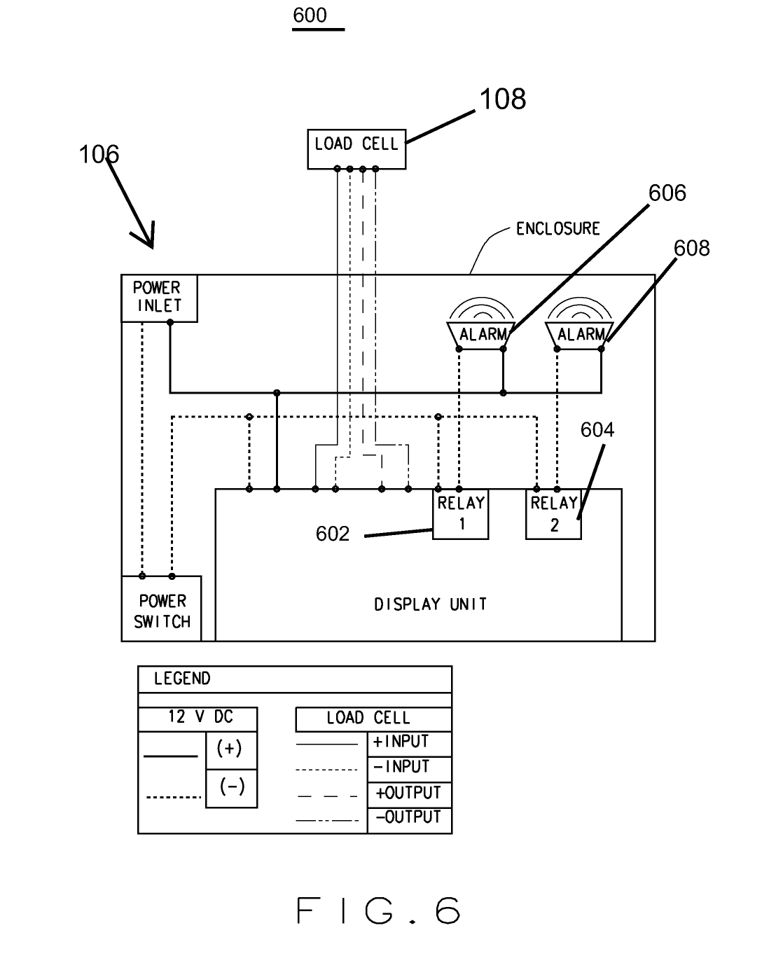

FIG. 6 is a schematic diagram of an exemplary circuit 600 for the injection pressure monitoring system showing the force transducer 108 coupled to a display unit circuit which includes two relay switches 602 and 604, and two relay-activated alarms 606 and 608. As shown in FIG. 6, connected to the relays in display unit 106 are two audible tone alarms, a first alarm that emits an intermittent tone, and a second alarm that emits a continuous tone. Display unit 106 is calibrated to trigger the first, intermittent alarm when the pressure sensor signals a first pressure limit of, for example, 80 psi, to provide a first warning to the operator that injection pressure is approaching the threshold level of 100 psi. Display unit 106 is further calibrated to trigger the second, continuous tone alarm when the pressure sensor signals a second pressure limit an injection pressure of 100 psi, indicating that pressure threshold has been reached. The injection pressure system is powered by a portable DC power source such as a 12VDC battery coupled to the display unit.

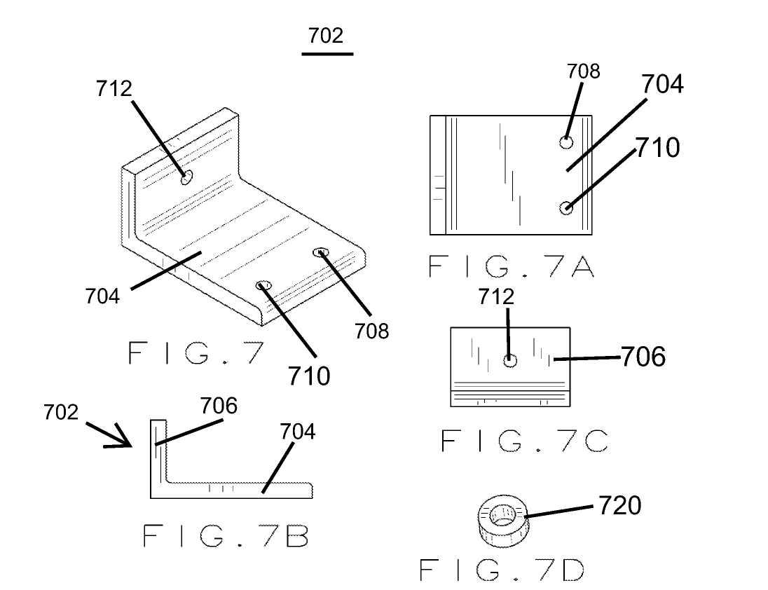

FIG. 7 is a drawing of an overhead side view of a calibration clamp 702 used to calibrate the injection pressure monitoring system. Calibration clamp 702 has a substantially L-shaped body, with a horizontal portion 704 coupled at a right angle to a vertical portion 706. FIG. 7A is a drawing of an overhead view of horizontal portion 704 of the calibration clamp 702. FIG. 7B is a drawing of a side view of the calibration clamp 702. FIG. 7C is a drawing of a side view of a vertical portion of the calibration clamp 702. FIG. 7D is a drawing overhead side view of one of two collars used together with the calibration clamp 702 in calibrating the injection pressure monitoring system, as described in further detail below. Calibration clamp 702 and collar 720 may be made using standard engineering design and machining techniques, also from any suitably rigid material that can be formed into the “angle-iron” shape, e.g., by injection molding from plastic or machining from metal sheet or block. An exemplary, non-limiting such material is aluminum.

An objective of the injection pressure monitoring device is to provide a convenient and reliable mechanism for controlling needle-tip pressure to avoid excessive injection pressures at the tissue site being treated. Calibration is thus performed to correlate a given force that is applied manually to the syringe assembly with a corresponding needle-tip pressure, such that compressive force as measured by force transducer 108 when the device is in use can then be used as an indirect measure of needle-tip pressure. Calibration may be performed using any instrument suitable for measuring pressures near atmospheric, such as a gauge or manometer, according to manufacturer's instructions, and using any fluid composition of any viscosity suitable for injection into tissue.

To prepare the injection pressure monitoring system for use, the system elements are assembled, together with calibration clamp 702 and collars 720, and the system calibrated. The operator collects the finger plate 112, syringe adapter 110, force transducer 108, calibration clamp 702, collars 720, and dispensing syringe 202. Preferably syringe 202 is a double-barreled syringe such as but not limited to the DuploJect syringe available from Baxter (Deerfield, Ill.), and this is coupled to blending connector 204, e.g. by pressure fit or luer-lock, as shown in FIG. 2. The syringe barrels are each loaded with 1 ml of water. A double-barreled syringe such as, for example, the DuploJect syringe, includes a plunger assembly having a cap coupling the two plungers arranged in parallel and each engaged within one of the barrels. The syringe assembly is connected to syringe adapter 110 by, for example, slidably engaging the dovetail projections 306 and 308 on the syringe-facing surface of syringe adapter 110 as shown in FIG. 3, with corresponding grooves in the cap of the plunger assembly. Force transducer 108 can be coupled to syringe adapter 110 by engaging a first threaded rod (not shown) with threaded opening 302 on syringe adapter 110, and fingerplate 112 similarly coupled to the opposing side of force transducer 108 by engaging a second threaded rod (not shown) with a threaded opening (not shown) in finger plate 112. It should be appreciated however that the methods for assembling the syringe, syringe adapter, load cell and finger plate will vary depending on the nature of the coupling methods used. For example, as described above, the syringe, syringe adapter, load cell and finger plate can be simply glued together using a medical-grade polymer adhesive, such as but not limited to cyanoacrylate polymer adhesive, silicone adhesive, and the like.

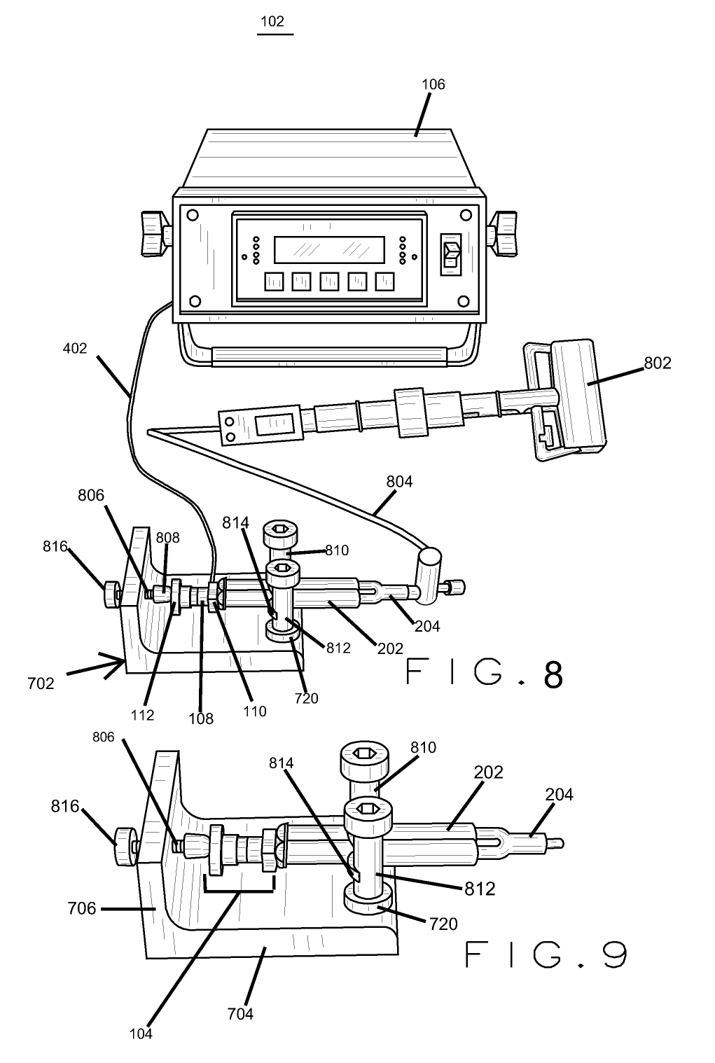

FIGS. 8 and 9 illustrate further aspects of the system as set up for calibration. FIG. 8 is a drawing of a complete calibration set-up for calibrating the injection pressure monitoring system, including an injection pressure monitoring assembly including the pressure sensor 104 coupled to display unit 106 and to a double-barreled syringe 202 positioned in calibration clamp 702, and a manometer 802 coupled to syringe 202 via a static fluid line 804. Calibration can performed, for example, using a handheld or portable manometer. A suitable manometer is a Monarch® Handheld Manometer as shown in FIG. 8 (Monarch® Digital Inflation Device, product #IN2130, available for example from Merit Medical OEM, South Jordan, Utah), but it should be readily appreciated that other manometers can be used.

FIG. 9 is a drawing of a close-up view of an injection pressure monitoring device including pressure sensor, adapter and finger plate, coupled to double-barreled syringe and positioned in the calibration clamp in preparation for calibration. Calibration clamp 702 is shown, substantially L-shaped, with horizontal wall 704 and vertical wall 706 joined at a right angle. A screw-driven ram 806 engages a suitably sized and threaded opening in vertical wall 706. A vinyl acorn nut 808 engages the leading end of screw-driven ram 806, and upon manual advancement of screw-driven ram 806, contacts finger plate 112 coupled to the force transducer 108. Two posts (810, 812), made of stainless steel for example, engage paired wings 814 on the syringe assembly 202, much as the fingers of an operator would normally do to hold the syringe body in place as force is manually applied to the plungers of the syringe. Posts 810 and 812 are for example substantially cylindrical and in any case suitably shaped and dimensioned to fit the curvature of the wings 814. One of two aluminum collars 720 is shown, each one fitting over one of posts 810 and 812, which collars are positioned under the wings of the syringe assembly to elevate the syringe assembly off of the body of the calibration clamp so as to align with the screw-driven ram. The aluminum body of the calibration clamp is generally sized to accommodate the posts, and a syringe such as the Baxter Duploject double barreled syringe assembly, together with the attached injection pressure measurement device including finger plate, pressure sensor and adaptor. A thumbscrew 816, in non-limiting example made from stainless steel, allows manual operation of screw-driven ram 806 to provide precise and constant force to finger plate 112 during calibration. It will be appreciated that force applied by the screw-driven ram 806 generates a pressure within the syringe barrels that is hydrostatically delivered as 10 psi of pressure within the manometer, as displayed on the manometer and can be verified by the a reading on display unit 106.

The fluid line is checked to ensure that no air is trapped in the fluid line. The manometer is activated. The pressure sensor 104 is coupled to the charged display unit 106 and the unit powered on. Display unit 106 is operated according to manufacturer's instructions for pressure calibration. For example, on the DCI 7600A Load Cell Display, front function keys are used to engage display unit 106 calibration menu. After ensuring that the load cell in pressure sensor 104 is reading at zero output (no external compression), the zero point calibration is entered by pressing ENTER. Span point calibration is performed by loading the dual syringe into the calibration clamp 702 as described above, and tightening using the thumbscrew to enforce contact, and then further tightening until the manometer reads 100 psi. Once the 100 psi reading stabilizes, the ENTER key on display unit 106 is pressed to store the full-scale output value.

Alarm points for the relay alarms, the time delay to trigger alarms, and decimal points for the digital read-outs are also set by following manufacturer's instructions for the particular display unit being used. On the DCI 7600A Load Cell Display, front function keys are used to set alarm points. For example, an entry of “+00750” sets a first limit at a pressure reading of 75 psi, and followed by an entry of “+00100” sets Channel#1 at a High Limit. Similarly, an entry of “+00950” sets a second limit at a pressure reading of 95 psi, and followed by another entry of “+00100” sets Channel#1 at a High Limit. An entry of, for example, “+00000” establishes a time delay of zero seconds before an audible alarm is triggered once the first pressure limit (i.e., for example, 75 psi) or the second pressure limit (i.e., for example, 95 psi) is reached. Front function keys are further used to set the decimal point position of the digital read-out. For example, a further entry of “+0100.0” sets the decimal point position accordingly. Thus, display unit 106 is calibrated to trigger the first alarm when the first pressure limit is reached, and to trigger the first alarm when the second pressure limit is reached.

It will be appreciated that the exact choice of time delay and the pressure level used to set the first and second limit may be varied, with the understanding that a pressure reading over a value of about 100 psi is to be avoided and the alarm limits are to be set to provide dual advance warning to the operator that pressure in the injection system is approaching 100 psi. For example, an operator may choose to set a first pressure limit at a pressure of 60 psi, and a second pressure limit at a pressure of 90 psi. Any pair of non-zero pressures up through about 100 psi may be used, according to the operator's preference as to the degree of advance warning before reaching a pressure of 100 psi. Preferably, two pressure values are selected, wherein the first is at least 50 psi, and the two values have a difference of at least 10 psi such that the second is at least 60 psi. Yet a highly experienced operator may, for example, choose to set the limits relatively close to one another and fairly high, e.g. a first limit at 90 psi and a second limit at 95 psi.

Following the above calibration of display unit 106, a calibration validation of pressure sensor 104 may be performed by ensuring that the applicator, e.g., double-barreled syringe 202, is tightened in calibration clamp 702 to enforce contact and then further tightened until the manometer reads 20 psi. Once the digital read-out on display unit 106 stabilizes, the pressure reading on display unit 106 is recorded. The tightening and recoding steps are repeated for each of multiple pre-selected pressure readings in the range of 0-100 psi, e.g., for 40 psi, 60 psi, 80 psi and 100 psi. The values recorded from display unit 106 for each selected pressure reading are then reviewed to determine if each value is within 5% of the manometer pressure reading. If not, the particular pressure sensor is not sufficiently accurate for adequate control of injection pressure.

Following calibration, calibration validation, and sterilization of the pressure sensor using a standard low-temperature sterilization technique such as gas-phase sterilization, the device and system are ready for use for monitoring injection pressure during treatment of an intervertebral disc in a subject, with a fluid therapeutic composition such as a cell-based composition, such as NuQu™ (ISTO Technologies, St. Louis, Mo.). A Display Unit such as the DCI 7600A Load Cell Display may first be charged using a supplied wall outlet adapter. Display unit 106 is then disconnected from the wall outlet if necessary, and positioned appropriately in the procedure room, for example within the operator's working area. The sterilized pressure sensor is presented to the sterile field. The fluid therapeutic composition is prepared as needed and the applicator filled with the composition. For example, each barrel of a dual-barreled syringe is filled with the two fluid components of NuQu™. The pressure sensor is then coupled to applicator by fitting the applicator adapter on the pressure sensor to the applicator, and applying manual pressure to the two pressure-fit pieces until they are firmly connected. The operator may then take hold of the syringe assembly including the pressure sensor, and the electrical connector coupled to the pressure sensor is coupled to the Display Unit.

Display unit 106 is powered on. A display unit such as the DCI 7600A Load Cell includes a function key for recording peak pressure reached and this may be pressed so that display unit 106 records the peak pressure reached during the procedure. The fluid composition is then delivered to the intervertebral disc by injection using the applicator. For example, a double-barreled syringe with a blending adapter as shown in FIGS. 2 and 8, coupled to a spinal needle and containing NuQu™ allows injection of NuQu™ directly into the nucleus pulposus of the intervertebral disc. Peak pressure as recorded during the procedure is noted, display unit 106 powered off and pressure sensor 104 disconnected. The pressure sensor may then be cleaned and sterilized according to standard medical protocol using low-temperature sterilization techniques.

### C. Kits

Kits according to the present disclosure may include one or more elements of the injection pressure monitoring device, and may further include components of a fluid therapeutic composition and/or reagents for preparing such a composition. A kit generally includes a package with one or more containers holding the device elements, composition components and any reagents, as one or more separate packages. The kit can also include other material(s) and tools which may be desirable from a user standpoint, such as any other material or tool useful in calibration or for cleaning, sterilizing or storing any component of the injection pressure monitoring device, including a calibration clamp. An exemplary kit contains an injection pressure monitoring device including a pressure sensor assembled with an adapter and finger plate, and a dispensing syringe, which is preferably a double-barreled syringe. The injection pressure monitoring device and syringe are preferably provided in sterilized packages. The kit may further include containers holding components of a dual component fluid therapeutic composition, such as for example NuQu™ (ISTO Technologies, Inc., St. Louis, Mo.). Kits according to the present disclosure preferably include instructions for assembling, calibrating and using the injection pressure monitoring sensor and system, and further for disassembling, cleaning, sterilizing and storing any components of the system. Instructions may further include those for preparing and delivering the fluid therapeutic composition using the pressure monitoring system. Instructions included in kits of the present disclosure can be affixed to packaging material or can be included as a package insert. While the instructions are typically written or printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

### D. Adaptations of the Methods of the Present Disclosure

By way of example, not of limitation, examples of the present invention shall now be given.

### EXAMPLE 1

### Injection Pressure Monitoring Device (IPMD)

The injection pressure device was initially tested with human cadaver lumbar intervertebral discs to demonstrate the safe injection pressure of an injectable (fluid) cell-based therapeutic composition known as NuQu™, available from ISTO Technologies, Inc, into an appropriately degenerated target disc. The device was also used to monitor injection pressures when NuQu™ was tested with 15 human subjects in Phase I clinical trials. Disc pressure during injection NuQu™, was achieved by monitoring the force applied to an injection syringe to maintain injection pressures below 100 psi. The system used was as shown in FIG. 1, an Injection Pressure Measurement Device (IPMD), including a display unit with limit-activated alarms and a pressure sensor that attaches to a standard Baxter 2 ml DuploJect syringe. The display unit, assembled by Design Concept Inc (DCI), was a Model 7600A-102 strain gauge readout mounted in a bench mount case along with the dual tone alarm. The unit was powered by an internal 12VDC battery. The removable Sensor was a Honeywell Model 11 (50 lbs max) load cell that connects to the back of the Display unit. The load cell was fitted with a comfortable Teflon thumb-plate on one end and a stainless steel syringe adapter on the other.

One skilled in the art would readily appreciate that the articles and kits described in the present disclosure are well adapted to carry out the objects and obtain the ends and advantages mentioned, as well as those inherent therein. The methods, procedures, treatments and kits described herein are merely representative and exemplary, and are not intended as limitations on the scope of the invention. It will be readily apparent to one skilled in the art that varying substitutions and modifications may be made to the present disclosure disclosed herein without departing from the scope and spirit of the invention.

All patents and publications mentioned in the specification are indicative of the levels of those skilled in the art to which the present disclosure pertains. All patents and publications are herein incorporated by reference to the same extent as if each individual publication was specifically and individually indicated to be incorporated by reference.

What is claimed is:

1. An injection pressure monitoring device for coupling to a dispensing syringe, comprising:
a. a pressure sensor comprising a miniature or subminiature load cell operable for measuring a compression load of up to about 100 lb, having an amplifier and an electrical coupling coupled thereto for coupling to a pressure display unit;
b. a syringe adapter coupled to the load cell, the syringe adapter having a first surface configured to engage an outward end portion of a plunger rod of a plunger rod assembly of the dispensing syringe; and
c. a finger plate coupled to the load cell.

2. The injection pressure monitoring device of claim 1, wherein the dispensing syringe is a double-barreled syringe having a plunger rod assembly comprising two plunger rods each having an outward end portion, and an end plate coupling the outward end portions of the two plunger rods, wherein the syringe adapter has a surface configured to engage the end plate of the plunger rod assembly.

3. The injection pressure monitoring device of claim 1, wherein the load cell comprises a subminiature load cell operable for measuring a compression load of up to about 50 lb.

4. The injection pressure monitoring device of claim 1, wherein the load cell comprises a compression load cell.

5. The injection pressure monitoring device of claim 1, wherein the finger plate comprises an injection molded finger plate.

6. The injection pressure monitoring device of claim 5, wherein the finger plate comprises polytetrafluoroethylene.

7. The injection pressure monitoring device of claim 5, wherein the finger plate has a contact surface having a central depression.

8. The injection pressure monitoring device of claim 5, wherein the finger plate has a contact surface having a finger groove.

9. An injection pressure monitoring system for maintaining injection pressure at a predetermined level in a tissue being injected with a fluid therapeutic composition from a dispensing syringe, the system comprising:
a. a pressure sensor for coupling to the syringe, comprising: (i) a miniature or subminiature load cell operable for measuring a compression load of up to about 100 lb, having an amplifier and an electrical coupling coupled thereto for coupling to a pressure display unit; (ii) a syringe adapter coupled to the load cell, the syringe adapter having a first surface configured to engage an outward end portion of a plunger rod of a plunger rod assembly of the dispensing syringe; and (iii) a finger plate coupled to the load cell;
b. a pressure display unit coupled to the pressure sensor electrical coupling and comprising a plurality of alarm elements; and
c. the dispensing syringe comprising a syringe body having at least one elongated barrel and a plunger rod assembly comprising a plunger rod having an outward end portion and operable for slidably engaging the inside of the elongated barrel, wherein the pressure sensor syringe adapter is coupled to the outward end portion of the plunger rod.

10. The injection pressure monitoring system of claim 9, wherein the dispensing syringe is a double-barreled syringe having a plunger rod assembly comprising two plunger rods each having an outward end portion, and an end plate coupling the outward end portions of the two plunger rods, wherein the syringe adapter has a surface configured to engage the end plate of the plunger rod assembly.

11. The injection pressure monitoring system of claim 9, wherein the pressure display unit comprises a first audible tone alarm and a second audible tone alarm.

12. The injection pressure monitoring system of claim 11, wherein the pressure display unit is configured to trigger the first audible tone alarm when the pressure sensor signals a pressure reading of at least 50 psi and to trigger the second audible tone alarm when the pressure sensor signals a pressure reading of at least 60 psi.

13. The injection pressure monitoring system of claim 11, wherein the pressure display unit is configured to trigger the first audible tone alarm when the pressure sensor signals a pressure reading of 75 psi and to trigger the second audible tone alarm when the pressure sensor signals a pressure reading of 95 psi.

14. The injection pressure monitoring system of claim 11, wherein the first audible tone alarm is an intermittent tone and the second audible tone is a continuous tone.

15. The injection pressure monitoring system of claim 11, further comprising a portable DC power source.

16. A kit for monitoring injection pressure in a dispensing syringe during injection of a fluid composition into tissue of a subject, the kit comprising:
a. a pressure sensor for coupling to the syringe, comprising: (i) a miniature or subminiature load cell operable for measuring a compression load of up to about 100 lb, having an amplifier and an electrical coupling coupled thereto for coupling to a pressure display unit; (ii) a syringe adapter coupled to the load cell, the syringe adapter having a first surface configured to engage an outward end portion of a plunger rod of a plunger rod assembly of the dispensing syringe; and (iii) a finger plate coupled to the load cell; and
b. a dispensing syringe having a plunger rod assembly including a plunger rod having an outward end portion.

17. The kit of claim 16, wherein the dispensing syringe is a double-barreled syringe having a plunger rod assembly comprising two plunger rods each having an outward end portion, and an end plate coupling the outward end portions of the two plunger rods, wherein the syringe adapter has a surface configured to engage the end plate of the plunger rod assembly.

18. The kit of claim 16, wherein the load cell comprises a subminiature load cell operable for measuring a compression load of up to about 50 lb.

19. The kit of claim 16, wherein the load cell comprises a compression load cell.

20. The kit of claim 16, wherein the finger plate comprises an injection molded finger plate.

21. The kit of claim 20, wherein the finger plate comprises polytetrafluoroethylene

22. The kit of claim 20, wherein the finger plate has a contact surface having a central depression.

23. The kit of claim 20, wherein the finger plate has a contact surface having a finger groove.
